(19) United States
(12) Patent Application Publication
(10) Pub. No.: US 2023/0337731 A1
(43) Pub. Date: Oct. 26, 2023

(54) MOUTHPIECE FOR AN AEROSOL GENERATING DEVICE

(71) Applicant: Philip Morris Products S.A., Neuchatel (CH)

(72) Inventors: Flavie ARAMENDY, Neuchatel (CH); Ricardo CALI, Mannheim (DE); Toney Moses RAJAN, Mannheim (DE); Yalcin AKPINAR, Neuchatel (CH); Alexandra SEREDA, Neuchatel (CH)

(73) Assignee: Philip Morris Products S.A., Neuchatel (CH)

(21) Appl. No.: 17/998,742

(22) PCT Filed: May 17, 2021
(86) PCT No.: PCT/EP2021/062936

§ 371 Date: Nov. 14, 2022

(30) Foreign Application Priority Data
May 18, 2020 (EP) 20175222.7

(51) Int. Cl. A24F 40/40; A24F 7/00; A24F 40/60; A24F 40/10; A24F 40/50; A61N 1/36

(52) U.S. Cl. CPC A24F 40/40; A24F 7/00; A24F 40/60; A24F 40/10; A24F 40/50; A61N 1/36

(57) ABSTRACT

A mouthpiece is provided for an aerosol generating device, including: an airflow channel extending at least partially through the mouthpiece to a mouthpiece opening arranged at a proximal end of the mouthpiece; a first electrode arranged on a front face of the mouthpiece to provide a first electrical contact to a user, the front face being a surface extending essentially perpendicular or angular to a longitudinal direction of the mouthpiece; and a second electrode arranged on a lateral surface of the mouthpiece to provide a second electrical contact to a user, the lateral surface being a peripheral surface of the mouthpiece. A method for an electrical stimulation of taste buds of a user, and a manufacturing method for a mouthpiece for an aerosol-generating device, are also provided.

The present disclosure relates to a mouthpiece for an aerosol generating device and an aerosol generating device with such a mouthpiece. The aerosol generating device can be understood as a smoking system having a liquid storage portion. The present disclosure further relates to a method for an electrical stimulation of taste buds of a user and a manufacturing method for a mouthpiece for an aerosol generating device.

Electrically heated smoking systems or aerosol generating devices of the prior art do have a number of advantages over conventional cigarettes and cigars, but there is still room for improvement in particular in view of a potential difference of aromatic notes and flavoring aspects when compared with a standard performance of conventional smoking articles, namely conventional cigarettes and cigars.

In more detail, an aerosol generating device generates aerosol for a user experience. An aerosol-generating substrate in such an aerosol generating device may comprise an e-liquid. The e-liquid is usually a liquid formulation containing propylene glycol, glycerin, nicotine, flavorings, and additives. A user of the aerosol generating device can customize an e-liquid formulation from a range of flavoring agents that are currently available in the market. The user can choose any of the flavoring agents such as to have an individual choice of user experience. However, a primary solvent used in e-liquids remains to be propylene glycol-glycerin concoction, which is organic polyalcohol. The nature of polyalcohol is that it has a certain degree of sweetness taste, which it imparts on to a user's tongue. This sweetness taste of the e-liquid is undesirable to the user, because she or he would expect the user experience to mimic a conventional smoking act.

Consequently, to overcome the issue of sweetness, an addition of chemicals may be added to the e-liquid to mask the sweetness. However, this would also mean that these chemicals would change the formulation of the e-liquid. The chemicals could also change the vaporization characteristics of the e-liquid compared to one without them, which could pose an additional challenge.

It is therefore an object of the invention to provide an improved mouthpiece for an aerosol generating device, which in particular reduces the issue of sweetness.

The object of the present disclosure is solved by the subject-matter of the independent claims, wherein further embodiments are incorporated in the dependent claims. It should be noted that the aspects of the disclosure described in the following apply to the mouthpiece for an aerosol generating device, the aerosol-generating device, the method for an electrical stimulation of taste buds of a user and the manufacturing method for a mouthpiece for an aerosol generating device.

According to an aspect of the present disclosure, there is provided a mouthpiece for an aerosol generating device. The mouthpiece comprises an airflow channel extending at least partially through the mouthpiece to a mouthpiece opening arranged at a proximal end of the mouthpiece.

The mouthpiece comprises a first electrode arranged on a front face of the mouthpiece to provide a first electrical contact to a user.

The mouthpiece comprises a second electrode arranged on a lateral surface of the mouthpiece to provide a second electrical contact to a user.

The present mouthpiece for an aerosol generating device may provide means to electrically stimulate the taste during consumption. It has a mouthpiece with two electrodes that can have a connection to a mouth of the user and can thereby stimulate the taste buds of the tongue via electrical signals. In other words, electrical impulses may be used to provide electrical taste stimulation on the tongue.

As a result, the present mouthpiece for an aerosol generating device may allow a better performance in view of aromatic notes and flavoring aspects. The stimulation via the electric potential may cause the user to experience a taste of saltiness, bitterness and/or sour to improve the flavor experience. The aromatic notes and flavoring aspects may provide flavor resembling the flavor of conventional cigarettes and cigars. This may ease a change of a user from conventional cigarettes and cigars to aerosol generating devices.

Further, the present mouthpiece for an aerosol generating device may allow reducing the above explained taste of sweetness due to the polyalcohol in the e-liquid. The two electrodes may be used to stimulate the taste buds of the tongue of the user to mask the sweetness of the polyalcohol. This may work without the above explained detrimental addition of chemical substances to the e-liquid, which would change the formulation of the e-liquid and lead to different vaporization characteristics.

The mouthpiece can be understood as a preferably replaceable mouthpiece for an aerosol generating device. The mouthpiece may comprise a reservoir configured for containing a liquid, namely an e-liquid. The mouthpiece may further comprise an aerosol generator as a heater configured for heating the reservoir and/or the liquid to form aerosol. The mouthpiece may be connected or reversibly connectable to a power source (e.g. a battery pack), wherein the power source provides power to the aerosol generator. In use, the liquid in the reservoir may be vaporized by the heater to form a supersaturated vapor. The supersaturated vapor may be mixed with and carried in an air flow. The air flow flows from an air inlet at a distal position or end to a proximal position or end, with the proximal position being a mouth end or a mouthpiece opening of the device. The air flow may enter the device at the distal position of the mouthpiece. During the flow, the vapor may condense to form an aerosol and the aerosol may be carried towards a mouth of a user. The mouthpiece may be cylindrical or tubular. The mouthpiece may have a circular, oval or other cross-sectional shape. The mouthpiece may comprise a mouthpiece housing. The mouthpiece housing may comprise an inner housing arranged in the mouthpiece housing. The mouthpiece and/or its housing(s) may surround an airflow channel.

The airflow channel can be understood as a channel configured to guide a flow of aerosol through the mouthpiece and to the user. The airflow channel may be surrounded by the mouthpiece. The airflow channel may extend at least partially in a longitudinal direction through the mouthpiece to a mouthpiece opening.

The mouthpiece opening can be understood to be configured to be inserted into a user's mouth. The mouthpiece opening may create an opening to be engaged by the mouth of a user. The mouthpiece opening can be arranged at a proximal end of the mouthpiece. The mouthpiece opening can be understood as an outlet of the airflow channel in a front face of a mouth end of the airflow channel.

The front face of the mouthpiece can be understood as an end surface of the mouthpiece. The front face may extend essentially perpendicular or angular to a longitudinal direction of the mouthpiece and/or the airflow channel. The front face may extend essentially perpendicular or angular to a lateral surface of the mouthpiece.

The lateral surface of the mouthpiece can be understood as different from the front face of the mouthpiece. It can be an outer surface of the mouthpiece extending along the longitudinal direction of the mouthpiece. The lateral surface can be a circumferential or peripheral surface of the mouthpiece.

The first electrode and the second electrode are different electrodes. They are positioned at different locations to provide different contact areas for the user. For example, a user may contact the first electrical contact with their tongue. For example, a user may contact the second electrode with another part of their tongue or their lip. A plane of the first electrode may be angular and preferably essentially perpendicular to a plane of the second electrode. The first electrode may extend over a part or the entire front face of the mouthpiece at the mouthpiece opening. The first electrode may additionally extend over a portion of the lateral surface of the mouthpiece. The second electrode may extend over a part of or the entire lateral surface of the mouthpiece. The electrodes may be made of an electric conductive material, preferably metal, and more preferably a Silver compound. The electrodes may have a dimension in a range of, for example, 20 mm×15 mm×0.2 mm.

In an embodiment, the first electrical contact on the front face of the mouthpiece may be accessible to a tip of a tongue of a user. This means the first electrical contact may be at a location where it is touched by the tip of the tongue and its taste buds, when in use. It may face the tip of the tongue. The first electrical contact may provide a first electric potential to the tip of the tongue, which is in particular sensitive for taste sensations (for example, salty tastes).

In an embodiment, the second electrical contact on the lateral surface of the mouthpiece may be accessible to a central area on the tongue of the user. This means the second electrical contact may be at a location where it is touched by a central part of the tongue, which is different to and “behind” the tip of the tongue, when in use. It essentially may face the central part of the tongue and its taste buds. The second electrical contact may provide a difference in electric potential between different parts of the tongue. A difference in potential between the first electric potential and the second electric potential may stimulate the taste buds of the tongue to bring an artificial taste to the tongue. For example, the second electrode may be available to an area of the tongue, which senses salty, sour or bitter flavors. In an embodiment, the second electrode is available to an area of the tongue other than the region that senses “sweet” flavors, to mask the sweetness of the polyalcohol.

In another embodiment, the second electrical contact on the lateral surface of the mouthpiece may be accessible to a lip of the user. This means the second electrical contact may be at a location where it is touched by a lip of the user, when in use. The second electrical contact may provide a second electric potential to the lip to provide a difference in potential between the first electric potential at the tongue and the second electric potential at the lip. The difference in electrical potential may stimulate the taste buds of the tongue to bring an artificial taste to the tongue to mask the sweetness of the polyalcohol.

In an embodiment, the second electrical contact on the lateral surface of the mouthpiece may be accessible to a lower lip of the user. In another embodiment, the second electrical contact on the lateral surface of the mouthpiece may be accessible to an upper lip of the user. In other words, the second electrical contact may be arranged and configured to touch the upper lip or the lower lip. The second electrical contact may also surround the mouthpiece at least partially to touch both lips.

In an embodiment, the first electrode may extend at last partially co-axially in the airflow channel. This means, the first electrode is not only at the front face of the mouthpiece, but may also extend in a longitudinal direction into and along the airflow channel. The first electrode may at least partially cover an inner surface of the airflow channel. Thereby, the first electrode is provided with a large surface that allows a cooling of the electrode, e.g. by means of the airflow.

In an embodiment, the second electrode may extend at last partially co-axially in the airflow channel. The second electrode may at least partially cover the inner surface of the airflow channel. Additionally or alternatively to the first electrode, also the second electrode may extend in a longitudinal direction into and along the airflow channel. Thereby, the second electrode is provided with a large surface that allows a cooling of the electrode, e.g. by means of the airflow. Preferably, the first electrode extends along the airflow channel in one portion of the airflow channel and the second electrode extends along the airflow channel in another portion of the airflow channel without contacting the first electrode.

In an embodiment, the second electrode may comprise an upper element arranged at an upper part of the mouthpiece body and a separate lower element arranged at an opposite, lower part of the mouthpiece body. This means the second electrode may comprise at least two parts or elements, which are arranged at different locations or portions of the mouthpiece body. As a result, the second electrode may be accessible for different body parts of the user, as e.g. an upper and a lower lip. This might intensify the user experience.

In an embodiment, the mouthpiece for an aerosol generating device may further comprise a third electrode arranged at the mouthpiece body to provide a third electrical contact. The third electrical contact may be accessible for a third region of the mouth, different from the first region and the second region. As a result, three different body parts of the user may be used for the taste stimulation, as e.g. the tongue, the upper and the lower lip or a first part of the tongue, a second, different part of the tongue and the lip. This might also intensify the user experience. Of course, the mouthpiece for an aerosol generating device may comprise even more than three electrodes.

In an embodiment, the mouthpiece may be reversibly connectable or connected to a power source. The power source may be configured to provide the electrical potential between the first electrode and the second electrode to stimulate the taste buds. The power source may be a battery or a battery pack. It may be rechargeable. The connection between mouthpiece and power source may be reversible.

In an embodiment, the electrical potential may be an alternating electrical potential. It may have a frequency in a range of 50 Hz to 1200 Hz, preferably 500 Hz to 700 Hz. Such frequencies may have an apparent effect on human tissues without causing vibrations of the electrode.

In an embodiment, the power source may be configured to provide the electrical potential with a current in a range of 20 μA to 300 μA, preferably 20 μA to 200 μA, more preferably 40 μA to 180 μA, and even more preferably 100 μA to 130 μA. The electrical current may be an alternating electrical current, preferably with steps of 20 μA to provide a distinguishable intensity of the stimulation.

In an embodiment, the mouthpiece may further comprise a temperature unit configured to control a temperature of the first electrode and/or the second electrode. The temperature unit may therefore comprise a means for adjusting the temperature of the electrodes, such as, for example, a heat sink. The heat sink may be a thermoelectric cooler, as e.g. a micropeltier element. It may have a dimension in a range of e.g. 1.0 mm×1.6 mm×0.62 mm to 6 mm×5.1 mm×0.62 mm. The heat sink may be arranged in contact with the first electrode and/or the second electrode. The heat sink may allow a heat transfer away from the first electrode and/or the second electrode to cool the first electrode and/or the second electrode. Cooling the first electrode and/or the second electrode may avoid an uncomfortable heat sensation at the tongue and/or the lip of the user.

In an embodiment, the heat transfer element may be configured for a physical contact with a housing of the mouthpiece. This contact may transfer the heat from the first electrode and/or the second electrode to the mouthpiece housing to cool the first electrode and/or the second electrode more efficiently. To provide enough thermal capacity for cooling the electrodes, the mouthpiece housing may be made of a dense material, which allows storing and dissipating heat energy, as for example, aluminum or ceramic. Additional heat sink materials may be utilized.

In an embodiment, the heat sink may comprises a thermo-paste. The thermo-paste may be arranged between the respective electrode and the mouthpiece housing. The thermo-paste may improve the heat transfer and thereby the cooling of the first electrode and/or the second electrode.

In an embodiment, the temperature unit may be configured to control the temperature of the first electrode and/or the second electrode in a range of 20 to 35 degrees Celsius, preferably 22 to 25 degrees Celsius. This temperature range seems to be very comfortable for the mouth of the user.

As a summary, the present mouthpiece may use electrical impulses with preferably alternating electric potential of varying frequency or varying current or both, optionally within a controlled temperature range, for taste stimulation. Current, frequency and/or temperature alone or in combination may result in a different taste and could reduce or eliminate the sweetness taste of e-liquids. This may be achieved without additional chemicals to mask the sweetness and would therefore not require any change to existing formulations of e-liquids.

According to another aspect of the present disclosure, there is provided an aerosol generating device. The aerosol generating device comprises a mouthpiece as described above, a power source and a control unit. The control unit and the power source are connectable to the mouthpiece.

The mouthpiece comprises an aerosol generator and a liquid reservoir. The aerosol generator is configured to heat liquid from the liquid reservoir to form an aerosol.

The power source is configured to provide an electrical potential between a first electrode and a second electrode of the mouthpiece.

The control unit is configured to control the electrical potential provided by the power source. The control unit can be part of the power source or part of the mouthpiece.

The present aerosol generating device therefore may provide means to electrically stimulate the taste during consumption. It has a mouthpiece with two electrodes that can have a connection to a mouth of the user to stimulate the taste buds of the tongue via electrical signals. The electrical signals may bring in artificial taste stimulation on the tongue.

As a result, the present aerosol generating device may deliver aromatic notes and flavoring aspects, which may ease a change of a user from conventional cigarettes and cigars to aerosol generating devices. The present aerosol generating device may further allow reducing or eliminating the issue in taste of sweetness due to the polyalcohol in the e-liquid by stimulating the taste buds of the tongue of the user to mask the sweetness of the polyalcohol.

In an embodiment, the power source may be additionally configured to provide energy to the aerosol generator. This means the power source may be the only power source of the aerosol generating device and it can be used to provide an electrical potential between the first electrode and the second electrode as well as to provide energy for heating the e-liquid to generate the aerosol. This makes the aerosol generating device very efficient and/or allows reducing the number of components. Of course, there can be also two different power sources for heating the e-liquid and for providing the electrical potential between the electrodes.

In an embodiment, the control unit may also be configured to control the amount of electrical potential between the first electrode and the second electrode. As a result, the control unit may thereby control an intensity or strength of the electrical stimulation. This may allow adapting the aerosol generating device to each and every user.

In an embodiment, the aerosol generating device may comprise a power supply housing, which houses the power source. The power supply housing may have a first portion at its outer surface configured for an overlapping interaction with a corresponding second portion arranged on the mouthpiece. As a result, it is easy to reliably attach or connect the power supply housing to the mouthpiece to form the aerosol generating device. The connection between the power supply housing and the mouthpiece may prevent a flow of fluids, including liquid and gas, between the power source and the mouthpiece. In embodiments, the power supply housing and the mouthpiece may be attached via any means known in the art including a fitting engagement or a complimentary lock-and-key type fitting, a screw attachment, friction fit, clasp, magnetic or other attachment.

In an embodiment, the aerosol generating device may comprise a button. The button may be configured to activate the aerosol generator. The button may be configured to activate an electrical potential between the first electrode and the second electrode. As a result, it may allow switching the aerosol generator and/or the artificial taste stimulation on and off.

In an embodiment, the button may be further configured to control the amount of electrical potential between the first electrode and the second electrode. As a result, it may further control an intensity of the electrical stimulation and thereby allow an adjustment of the strength of taste simulation.

As a result, the present aerosol generating device may allow a taste improvement by electrical stimulation during consumption. It may further allow a customizable electrical stimulation experience by adjusting a taste intensity by the user and/or a by means of a preset based on a tracked historic usage of the device. The present aerosol generating device may further allow a proper temperature of the contact areas with the user by means of a temperature control of the electrodes via a heat transfer element that uses a housing of the aerosol generator to dissipate the heat. It may still allow a specific position of the electrodes to ensure an adequate and ergonomic functioning.

According to another aspect of the present disclosure, also a method for an electrical stimulation of taste buds of a user is presented. The method for an electrical stimulation of taste buds of a user comprises the following steps, not necessarily in this order:
providing a mouthpiece as described above, and
providing an electrical potential between a first electrode and a second electrode of the mouthpiece to stimulate taste buds on a tongue of the user.

The present method for an electrical stimulation of taste buds may provide means to stimulate the taste electrically during consumption. It may allow a better performance in view of aromatic notes and flavoring aspects. The present method for an electrical stimulation may further allow reducing the taste of sweetness due to the polyalcohol in an e-liquid by masking this sweetness by the electrical stimulation.

According to another aspect of the present disclosure, there is provided a manufacturing method for a mouthpiece. The manufacturing method for a mouthpiece comprises the following steps, not necessarily in this order:
providing a mouthpiece (initial or raw mouthpiece element), which comprises an airflow channel extending at least partially through the mouthpiece to a mouthpiece opening arranged at a proximal end of the mouthpiece,
arranging a first electrode on a front face of the mouthpiece to provide a first electrical contact to a user, and
arranging a second electrode on a lateral surface of the mouthpiece to provide a second electrical contact to a user (and to provide the final or finished mouthpiece).

The present manufacturing method for a mouthpiece may provide a mouthpiece to stimulate the taste electrically during consumption. The manufacturing method is easy to implement and leads to a reliable and safe mouthpiece. The mouthpiece allows a better performance in view of aromatic notes and flavoring aspects. The mouthpiece may allow reducing the taste of sweetness due to the polyalcohol in an e-liquid by means of an electrical stimulation.

Below, there is provided a non-exhaustive list of non-limiting examples. Any one or more of the features of these examples may be combined with any one or more features of another example, embodiment, or aspect described herein.
A. A mouthpiece for an aerosol generating device comprising:
   an airflow channel extending at least partially through the mouthpiece to a mouthpiece opening arranged at a proximal end of the mouthpiece,
   a first electrode arranged on a front face of the mouthpiece to provide a first electrical contact to a user, and
   a second electrode arranged on a lateral surface of the mouthpiece to provide a second electrical contact to a user.
B. Mouthpiece according to claim A, wherein the first electrical contact on the front face of the mouthpiece is accessible to a tip of a tongue of a user.
C. Mouthpiece according to claim A or B, wherein the second electrical contact on the lateral surface of the mouthpiece is accessible to a central area on the tongue of the user.
D. Mouthpiece according to claim A or B, wherein the second electrical contact on the lateral surface of the mouthpiece is accessible to a lip of the user.
E. Mouthpiece according to claim D, wherein the second electrical contact on the lateral surface of the mouthpiece is accessible to a lower lip of the user.
F. Mouthpiece according to claim D, wherein the second electrical contact on the lateral surface of the mouthpiece is accessible to an upper lip of the user.
G. Mouthpiece according to one of the preceding claims, wherein the first electrode extends at last partially co-axially in the airflow channel.
H. Mouthpiece according to one of the preceding claims, wherein the second electrode extends at last partially co-axially in the airflow channel.
I. Mouthpiece according to one of the preceding claims, wherein the second electrode comprises an upper element arranged at an upper part of the mouthpiece body and a separate lower element arranged at an opposite, lower part of the mouthpiece body.
J. Mouthpiece according to one of the preceding claims, further comprising a third electrode arranged at the mouthpiece body to provide a third electrical contact which is accessible for a third region of the mouth, different from the first region and the second region.
K. Mouthpiece according to one of the preceding claims, comprising or connectable to a power source configured to provide an electrical potential between the first electrode and the second electrode.
L. Mouthpiece according to the preceding claim, wherein the electrical potential is an alternating electric-potential with a frequency in a range of 50 Hz to 1200 Hz.
M. Mouthpiece according to claim K or L, wherein the power source is configured to provide the electrical potential with a current in a range of 20 μA to 300 μA.
N. Mouthpiece according to one of the preceding claims, further comprising a temperature unit configured to control a temperature of the first electrode and/or the second electrode, wherein the temperature unit comprises at least a heat sink, which is arranged in contact with the first electrode and/or the second electrode.
O. Mouthpiece according to the preceding claim, wherein the heat sink comprises a thermo-paste.
P. Mouthpiece according to claim N or O, wherein the heat transfer element is configured for a contact with a mouthpiece housing of the mouthpiece.
Q. Mouthpiece according to one of the claims N to P, wherein the temperature unit is configured to control the temperature of the first electrode and/or the second electrode in a range of 20 to 35 degrees Celsius.
R. An aerosol generating device, comprising:
   a mouthpiece according to one of the preceding claims,
   a power source, and
   a control unit,
   the mouthpiece comprises an aerosol generator and a liquid reservoir, wherein the aerosol generator is configured to heat liquid from the liquid reservoir to form aerosol, the power source is configured to provide an electrical potential between a first electrode and a second electrode of the mouthpiece,
   the control unit is configured to control the electrical potential provided by the power source, and
   the control unit and the power source are connectable to the mouthpiece.
S. Aerosol generating device according to the preceding claim, wherein the power source is also configured to provide energy to the aerosol generator for heating the aerosol.
T. Aerosol generating device according to one of the claims R to S, wherein the control unit is also configured to control the amount of electrical potential between the first electrode and the second electrode and thereby control an intensity of the electrical stimulation.
U. Aerosol generating device according to one of the claims R to T, wherein a power supply housing of the power source comprises a first portion configured for an overlapping interaction with a correspondingly shaped second portion arranged at the mouthpiece.
V. Aerosol generating device according to one of the claims R to U, further comprising a button configured to activate the aerosol generator and/or the electrical potential between the first electrode and the second electrode.
W. Aerosol generating device according to the preceding claim, wherein the button is further configured to control the amount of electrical potential between the first electrode and the second electrode and thereby control an intensity of the electrical stimulation.
X. A method for an electrical stimulation of taste buds of a user, comprising:
   providing a mouthpiece according to one of the claims A to Q, and
   providing an electrical potential between a first electrode and a second electrode of the mouthpiece to stimulate taste buds on a tongue of the user.
Y. A manufacturing method for a mouthpiece for an aerosol generating device, comprising:
   providing a mouthpiece, which comprises an airflow channel extending at least partially through the mouthpiece to a mouthpiece opening arranged at a proximal end of the mouthpiece,
   arranging a first electrode on a front face of the mouthpiece to provide a first electrical contact to a user, and
   arranging a second electrode on a lateral surface of the mouthpiece to provide a second electrical contact to a user.

Examples will now be further described with reference to the figures in which:

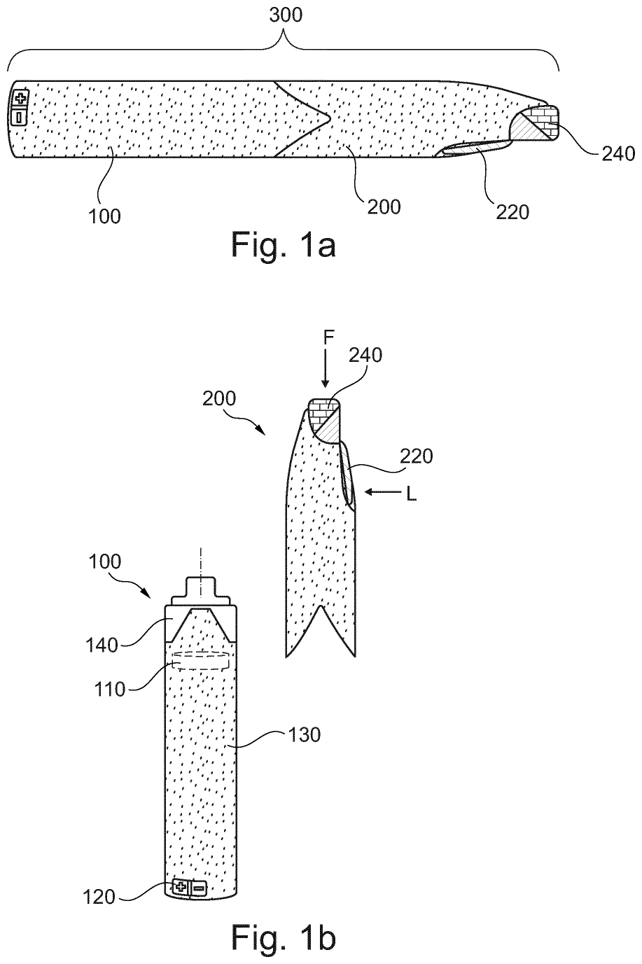

FIGS. 1a and 1b show schematically and exemplarily an embodiment of an aerosol generating device according to the disclosure.

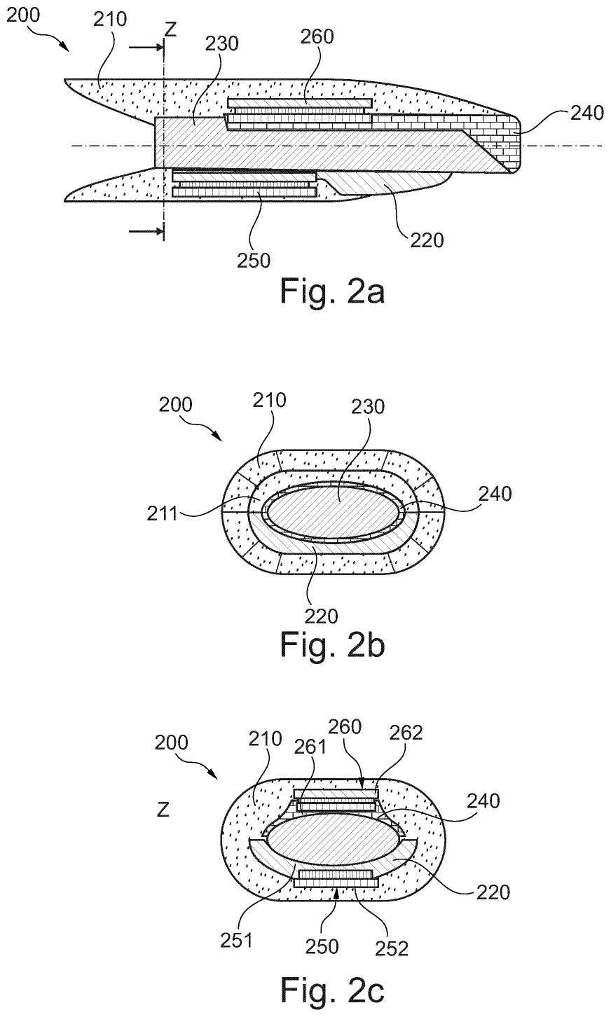

FIGS. 2a, 2b and 2c show schematically and exemplarily an embodiment of a mouthpiece for the aerosol generating device according to the disclosure.

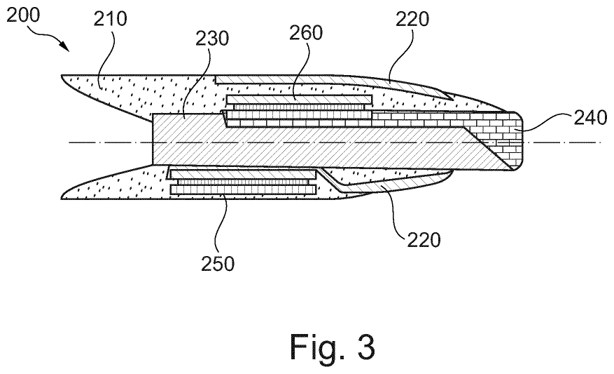

FIG. 3 shows schematically and exemplarily another embodiment of a mouthpiece for the aerosol generating device according to the disclosure.

FIGS. 1a and 1b show an aerosol generating device 300 according to the present disclosure. The aerosol generating device 300 comprises a power supply housing 100 and a mouthpiece 200. In FIG. 1a, the power supply housing 100 and the mouthpiece 200 are assembled; in FIG. 1b, the power supply housing 100 and the mouthpiece 200 are disassembled into separate parts.

The mouthpiece 200 comprises an aerosol generator (not shown) and a liquid reservoir (not shown). The aerosol generator is configured to heat liquid from the liquid reservoir to form an aerosol.

The mouthpiece 200 comprises a first electrode 240 arranged on a front face F of the mouthpiece 200 to provide a first electrical contact to a user. The mouthpiece 200 comprises a second electrode 220 arranged on a lateral surface L of the mouthpiece 200 to provide a second electrical contact to a user. When in use, a tongue of the user makes contact with the first electrode 240 and, for example, a lower lip is in contact with the second electrode 220. The mouthpiece 200 comprises an airflow channel 230 (see FIGS. 2a-c) extending through the mouthpiece 200.

The power supply housing 100 comprises a power source 130 and a control unit 110. The power supply housing 100 and thereby the control unit 110 and the power source 130 are reversibly connectable to the mouthpiece 200.

The power source 130 comprises a battery configured to provide an electrical potential between the first electrode 240 and the second electrode 220 of the mouthpiece 200.

The control unit 110 is configured to control the electrical potential provided by the power source 130. The control unit 110 and the two electrodes 220, 240 form an electrical circuit. If the user touches the electrodes 220, 240 with her or his lips or tongue, the circuit is completed between both electrodes 220, 240 (through the user) and this is recognized by the control unit 110. The control unit 110 subsequently starts providing an artificial taste stimulation. The control unit 110 may also control an intermittent operation of the taste stimulation to optimise a taste sensitisation.

On activation of the aerosol generator device 300 by the user, the aerosol generation occurs and the electrodes 240, 220 on the mouthpiece 200 receive the electrical potential. The electrical potential and a current between the first electrode 240 and the second electrode 220 act on the user's tongue with a simulation of an artificial taste based on the potential and/or the current. The preferably alternating current is here in a range of 20 μA to 200 μA with steps of 20 μA and provides a distinguishable intensity of the stimulation. The frequency of the alternating electric potential is here in the range of 50 Hz to 1200 Hz.

The power supply housing 100 further comprises a button 120, which can be pressed to switch the aerosol generating device 300 on or off, for example when it is pressed for a longer duration. The button 120 can also be pressed during the operation of the aerosol generating device 300 in an intermittent manner to control an intermittent operation of the taste stimulation to optimise a taste sensitisation. The control via the button 120 can be alternatively or additionally to the control via the control unit 110. The control unit 110 may store a preferred setting in an internal memory to adjust on start an intensity of the taste stimulation according to a preferred setting of the user.

The power supply housing 100 further comprises first portions 140, which are in physical contact and overlapping with respective second portions on the mouthpiece 200 to allow a reliable mechanical connection between the power supply housing 100 and the mouthpiece 200.

FIGS. 2a to 2c show the mouthpiece 200 according to the present disclosure. FIG. 2a shows a cross section through the mouthpiece 200 in a side view. FIG. 2b shows a cross section through the mouthpiece 200 in a front view. FIG. 2c shows a cross section through the mouthpiece 200 in a rear view along section Z-Z in FIG. 2a.

As stated to FIGS. 1a and 1b, the mouthpiece 200 surrounds the airflow channel 230 through the mouthpiece 200. At the mouthpiece 200, the two electrodes 240, 220 for an electric-stimulation are arranged. The first electrode 240 here extends as a long part from the front face F co-axially along a longitudinal axis of the air-channel 230. The second electrode 220 is arranged on the lateral surface L of the mouthpiece 200.

The first electrode 240 is in contact with a thermoelectric cooling element 260 using thermo-paste on a first side 261 and a second side 262. The second electrode 220 is in contact with another thermoelectric cooling element 250 using thermo-contact paste on another first side 251 and another second side 251. Both of these thermoelectric cooling elements 250, 260 have contact with a mouthpiece housing 210 of the mouthpiece 200, such that a heat transfer is efficient between the electrodes 240, 220 and the mouthpiece housing 210 to maintain a suitable temperature on the electrodes 240, 220. Further, the mouthpiece 200 and the mouthpiece housing 210 transfers heat over overlapping or tapered portions to corresponding overlapping or tapered portions 140 of the heat-dissipating power supply housing 100. The temperature range for the operation of the aerosol generator device 300 is here between 24° C. to 35° C.

The mouthpiece housing 210 comprises here an inner housing 211 also having a heating conduction ability. The mouthpiece housing 210 and the inner housing 211 are here oval-tubular and create the air channel 230 in the mouthpiece 200.

FIG. 3 shows another embodiment of the mouthpiece 200 according to the present disclosure in a lateral cross section. This mouthpiece 200 is modified with a second electrode 220 in an extended manner such that both lips (upper and lower lips) of the user can touch the second electrode 220 while the first electrode 240 still makes contact with the tongue. The second electrode 220 therefore comprises an upper and a lower element.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A±20% of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed disclosure. Also, all ranges include the maximum and minimum points disclosed and include any intermediate ranges therein, which may or may not be specifically enumerated herein.

Although illustrative examples of the present disclosure have been described above, in part with reference to the accompanying drawings, it is to be understood that the disclosure is not limited to these examples. Variations to the disclosed examples can be understood and effected by those skilled in the art in practicing the disclosure, from a study of the drawings, the specification and the appended claims.

In the claims, any reference signs placed between parentheses shall not be construed as limiting the claim. The term “comprising” does not exclude the presence of elements or steps other than those listed in a claim. The word “a” or “an” preceding an elements does not exclude the presence of a plurality of such elements. The disclosure can be implemented by means of hardware comprising several distinct elements. In the device claim enumerating several means, several of these means can be embodied by one and the same item of hardware. The mere fact that certain measured are recited in mutually different dependent claims does not indicate that a combination of these measure cannot be used to advantage.

1.-15. (canceled)

16. A mouthpiece for an aerosol generating device, comprising:
an airflow channel extending at least partially through the mouthpiece to a mouthpiece opening arranged at a proximal end of the mouthpiece;
a first electrode arranged on a front face of the mouthpiece to provide a first electrical contact to a user, the front face being a surface extending essentially perpendicular or angular to a longitudinal direction of the mouthpiece; and
a second electrode arranged on a lateral surface of the mouthpiece to provide a second electrical contact to a user, the lateral surface being a peripheral surface of the mouthpiece.

17. The mouthpiece according to claim 16, wherein the first electrical contact on the front face of the mouthpiece is accessible to a tip of a tongue of a user.

18. The mouthpiece according to claim 16, wherein the second electrical contact on the lateral surface of the mouthpiece is accessible to a central area on the tongue of the user.

19. The mouthpiece according to claim 16, wherein the second electrical contact on the lateral surface of the mouthpiece is accessible to a lip of the user.

20. The mouthpiece according to claim 16, wherein the second electrode comprises at least two elements, which are arranged at different locations of the mouthpiece to be accessible for different body parts of the user.

21. The mouthpiece according to claim 16, wherein the mouthpiece is reversibly connectable to a power source configured to provide an electrical potential between the first electrode and the second electrode.

22. The mouthpiece according to claim 21, wherein the power source is further configured to provide the electrical potential as an alternating electrical potential with a frequency in a range of 50 Hz to 1200 Hz.

23. The mouthpiece according to claim 21, wherein the power source is further configured to provide the electrical potential with a current in a range of 20 μA to 300 μA.

24. The mouthpiece according to claim 16,
further comprising a temperature unit configured to control a temperature of the first electrode and/or the second electrode,
wherein the temperature unit comprises at least a heat sink, which is arranged in contact with the first electrode and/or the second electrode.

25. The mouthpiece according to claim 24, wherein the heat sink comprises a thermo-paste.

26. The mouthpiece according to claim 25, wherein the temperature unit is further configured to control the temperature of the first electrode and/or the second electrode in a range of 20 degrees Celsius to 35 degrees Celsius.

27. An aerosol-generating device, comprising:
a mouthpiece according to claim 16;
a power source; and
a control unit,
wherein the mouthpiece comprises an aerosol generator and a liquid reservoir,
wherein the aerosol generator is configured to heat liquid from the liquid reservoir to form aerosol,
wherein the power source is configured to provide an electrical potential between a first electrode and a second electrode of the mouthpiece,
wherein the control unit is configured to control the electrical potential provided by the power source, and
wherein the control unit and the power source are connectable to the mouthpiece.

28. The aerosol-generating device according to claim 27, further comprising a power supply housing comprising a first portion configured for an overlapping interaction with a correspondingly shaped second portion arranged at the mouthpiece.

29. A method for an electrical stimulation of taste buds of a user, comprising:
providing a mouthpiece according to claim 16; and
providing an electrical potential between a first electrode and a second electrode of the mouthpiece to stimulate taste buds on a tongue of the user.

30. A manufacturing method for a mouthpiece for an aerosol-generating device, comprising:
providing a mouthpiece, which comprises an airflow channel extending at least partially through the mouthpiece to a mouthpiece opening arranged at a proximal end of the mouthpiece;
arranging a first electrode on a front face of the mouthpiece to provide a first electrical contact to a user, the front face being a surface extending essentially perpendicular or angular to a longitudinal direction of the mouthpiece; and
arranging a second electrode on a lateral surface of the mouthpiece to provide a second electrical contact to a user, the lateral surface being a peripheral surface of the mouthpiece.
